(19) United States
(12) Patent Application Publication
(10) Pub. No.: US 2025/0194906 A1
(43) Pub. Date: Jun. 19, 2025

(54) OVER THE SCOPE BIFURCATION ASSEMBLY WITH CLIP RELEASE MECHANISM

(71) Applicants: BOSTON SCIENTIFIC MEDICAL DEVICE LIMITED, Galway (IE); BOSTON SCIENTIFIC SCIMED, INC., Maple Grove, MN (US)

(72) Inventors: Austin Grant JOHNSON, Worcester, MA (US); Rajivkumar SINGH, Pune (IN); Colby HARRIS, Norfolk, MA (US); Jennifer LEWITZKY, Lexington, MA (US)

(21) Appl. No.: 18/962,980

(22) Filed: Nov. 27, 2024

Related U.S. Application Data
(60) Provisional application No. 63/612,158, filed on Dec. 19, 2023.

(51) Int. Cl. A61B 1/00; A61F 2/856

(52) U.S. Cl. CPC A61B 1/0014; A61F 2/856

(57) ABSTRACT

A system includes a cap, a clip having jaws movable between open and closed configurations, a tubing system, and a control assembly. The cap includes proximal and distal portions. The tubing system includes a proximal structure and distal structures. A proximal end of each of the structures is connected to a distal end of the structure via a bifurcation such that the structures are in communication with the structure. The distal end of each of the structures is connected to the cap. The assembly includes wires extending through the structure and a corresponding one of the structures to be releasably coupled to the clip, and overtubes received within the structures to extend over a portion of the wires. The wires and the overtubes longitudinally moves relative to the cap to move the clip between insertion, closed and review configurations.

### PRIORITY CLAIM

The present disclosure claims priority to U.S. Provisional Patent Application Ser. No. 63/612,158 filed Dec. 19, 2023; the disclosure of which is incorporated herewith by reference.

### FIELD

The present disclosure relates to endoscopic devices and, in particular, relates to endoscopic clipping devices for treating tissue along the gastrointestinal tract.

### BACKGROUND

Physicians have become more willing to perform aggressive interventional and therapeutic endoscopic gastrointestinal (GI) procedures, which may increase the risk of perforating the wall of the GI tract or may require closure of the GI tract wall as part of the procedure. Such procedures may include, for example, the removal of large lesions, tunneling under the mucosal layer of the GI tract to treat issues below the mucosa, full thickness removal of tissue, treatment of issues on other organs by passing outside of the GI tract, and endoscopic treatment/repair of post-surgical issues (e.g., post-surgical leaks, breakdown of surgical staple lines, and anastomotic leaks).

Currently, tissue may be treated via endoscopic closure devices including through-the scope clips or over-the-scope clips. Over-the-scope clips may be particularly useful for achieving closure of larger tissue defects. These endoscopic closure devices can save costs for the hospital and may provide benefits for the patient. In some cases, however, current endoscopic closure devices may be difficult to use, time consuming to position, or insufficient for certain perforations, conditions and anatomies. For example, current over-the-scope clips generally require launching of the clip from a position in which the clip itself is not visible to the operator. That is, prior to clipping the operator may view the target tissue to be clipped and, based on this visualization of the target tissue may determine that the distal end of the device and the clip are in a desired position relative to the target tissue. Based on the observation of the target tissue, the operator then deploys the clip without being able to see the clip itself until it is deployed. Once deployed, such current over-the scope clips are generally incapable of being repositioned.

### SUMMARY

The present disclosure relates to a clipping system for treating tissue. The clipping system includes an end cap including a proximal portion configured to be coupled to a distal end of an insertion device and a distal portion extending distally from the proximal portion. The clipping system also includes a clip including first and second jaws movable relative to one another between an open configuration, in which the first and second jaws are separated from one another, and a biased closed configuration, in which the first and second jaws are moved toward one another, the clip configured to be mounted over the distal portion of the end cap in the open configuration.

In addition, the clipping system includes a bifurcated tubing system including a single proximal tubular structure extending from a proximal end to a distal end and a pair of distal tubular structures, each of which extends from a proximal end to a distal end, the proximal end of each of the distal tubular structures connected to the distal end of the single proximal tubular structure via a bifurcation such that the pair of distal tubular structures are in communication with the single proximal tubular structure, the distal end of the each of the distal tubular structures connected to the end cap. Furthermore, a control assembly including a pair of control wires, each of which extends through the single proximal tubular structure and a corresponding one of the distal tubular structures to be releasably coupled to the clip, and a pair of overtubes received within the distal tubular structures to extend over a portion of the control wires received therein, the control wires and the overtubes configured to interface with one another and be longitudinally movable relative to the end cap to move the clip between an insertion configuration, in which the clip is mounted over the end cap in the open configuration, the closed configuration, in which the clip is moved distally off of the end cap to grip tissue therebetween, and a review configuration, in which the clip is physically separated from the end cap to enhance visual observation of the clip.

In an embodiment, the proximal portion of the end cap includes a pair of longitudinally extending grooves aligned with the distal tubular structures, a spacer received within each of the grooves and held thereagainst via an O-ring.

In an embodiment, the spacers are movable relative to the end cap from a first position, in which the overtubes is slidably receivable between the grooves and the spacers, and a second position, in which the O-ring compresses the spacer radially inward upon movement of the overtubes distally therepast so that openings formed between the spacers and the grooves are sized, shaped and configured to prevent proximal receipt of the overtubes therein.

In an embodiment, each of the overtubes include a first stop configured to engage a distal end of a corresponding one of the spacers when the corresponding one of the spacers is in the second position and a second stop configured to interface with a portion of the corresponding one of the control wires.

In an embodiment, each of the control wires includes a pusher element protruding from the control wire, the control wire received within the corresponding overtube so that the pusher element is positioned proximally of the second stop so that the pusher element engages the stop as the control wire is moved distally relative to the end cap to correspondingly move the overtube distally relative to the end cap.

In an embodiment, each of the control wires including a protruding element along a portion of the control wire, the control wire received within the corresponding overtube so that the protruding element is positioned distally of the second stop to prevent the corresponding overtube from slipping thereoff of subsequent to a deployment of the clip.

In an embodiment, the one of the pusher element and the protruding element is configured as a hypotube crimped over a portion of the control wire.

In an embodiment, each of the control wires is releasably coupled to a corresponding one of the first and second jaws of the clip via an enlarged distal end received within a yoke of the corresponding one of the first and second jaws, the enlarged distal end connected to a remaining length thereof via a joint configured to separate the enlarged distal end from the remaining length when subject to a force exceeding a predetermined threshold value.

In an embodiment, each of the enlarged distal ends has a cross-sectional area that is larger than a cross-sectional area of an opening through which it enters the yoke of the corresponding jaw.

In addition, the present disclosure relates to a clipping system for treating tissue. The clipping system includes an end cap including a proximal portion couped to a distal end of an endoscope and a distal portion extending distally from the proximal portion. The clipping system also includes a clip mounted over the distal portion of the end cap so that first and second jaws of the clip are separated from one another toward an open configuration, the clip biased toward a closed configuration, in which the first and second jaws are moved toward one another.

In addition, the clipping system includes a bifurcated tubing system including a single proximal tubular structure and a pair of distal tubular structures, the single proximal tubular structure extends along a majority of a length of the endoscope from a proximal end connected to a user interface at a proximal end of an insertion device to a distal end, proximal ends of the distal tubular structures connected to the distal end of the single proximal tubular structure via a bifurcation such that the distal tubular structures are in communication with the single proximal tubular structure and distal ends of the distal tubular structures connected to the end cap.

The clipping system includes a control assembly including a pair of control wires and a pair of overtubes, the control wires extending through the single proximal tubular structure and the distal tubular structures so that distal ends of the control wires extend distally past the distal ends of the distal tubular structures to be releasably coupled to the clip, the overtubes received within the distal tubular structures to extend over portions of the control wires received therein, the control wires and the overtubes interfacing with one another and movable relative to the insertion device to move the clip relative to the end cap between an insertion configuration, in which the clip is mounted over the end cap, the closed configuration, in which the clip is slid off of the end cap to grip tissue between the first and second jaws thereof, and a review configuration, in which the clip is physically separated from the end cap to enhance visual observation of the clip.

In an embodiment, the proximal portion of the end cap includes a pair of longitudinally extending grooves aligned with the distal tubular structures, spacers received within the grooves and held thereagainst via O-rings which moves the spacers from a first position, in which the overtubes is slidably receivable between the grooves and the spacers, and a second position, in which the O-rings compresses the spacers radially inward upon movement of the overtubes distally therepast so that openings formed between the spacers and the grooves are sized, shaped and configured to prevent proximal receipt of the overtubes therein.

In an embodiment, each of the overtubes include a first stop configured to engage a distal end of a corresponding one of the spacers when the corresponding one of the spacers is in the second position and a second stop configured to interface with a portion of the corresponding one of the control wires.

In an embodiment, each of the control wires includes a pusher element protruding from the control wire, the control wire received within the corresponding overtube so that the pusher element is positioned proximally of the second stop so that the pusher element engages the stop as the control wire is moved distally relative to the end cap to correspondingly move the overtube distally relative to the end cap.

In an embodiment, the system further includes a user interface including a handle shaft connected to a proximal end of the insertion device and a slider slidably mounted over the handle shaft and connected to proximal end of the control wires so that a longitudinal movement of the slider relative to the handle shaft correspondingly moves the control wires relative to the endoscope.

In an embodiment, the user interface further includes a lock movable between the first position, in which the slider is prevented from being moved further distally relative to the handle shaft to prevent movement of the clip from the review configuration toward a deployed configuration, and the second position, in which the slider is distally movable relative to the handle shaft to initiate the deployed configuration in which the overtube is moved distally past the spacer.

In addition, the present disclosure relates to a method for treating tissue. The method includes inserting a clip to a target area in a body lumen via an endoscope, the clip mounted over a distal end of the endoscope, via an adapter, in an open insertion configuration in which jaws of the clip are separated from one another; drawing tissue into a channel of the adapter and between the jaws of the clip; moving the clip from the open insertion configuration toward a biased closed configuration by releasing a tension along a pair of control wires, distal ends of which are releasably coupled to the jaws of the clip, the control wires extending through a bifurcated tubing assembly including a single proximal tubular structure extending along a majority of a length of the endoscope and a pair of distal tubular structures connected to the single proximal tubular structure via a bifurcation; moving the clip from the closed configuration toward a review configuration by moving the clip distally relative to the adapter so that the clip is spaced therefrom and visible via a viewing system of the endoscope, the clip moved toward the review configuration via overtubes moved distally relative to the endoscope to be pressed distally against a surface of the clips, the overtubes received within the distal tubular structures and extending over portions of the control wires received therein, interfacing therewith such that a distal motion of the control wires relative to the endoscope correspondingly moves the overtubes distally relative to the endoscope; and determining whether the clip is clipped over a target tissue, as desired.

In an embodiment, when a user determines that the clip requires repositioning, drawing the control wires proximally relative to the endoscope to draw the clip toward an end cap and over a distal portion thereof to the open insertion configuration.

In an embodiment, when a user determines that the clip is clipped over the target tissue, initiating a deployment of the clip by moving the overtubes further distally relative to the endoscope so that the overtubes are moved distally past spacers housed against portions of an end cap via O-rings, the O-rings compressing the spacers radially so that openings formed between the spacers and are sized, shaped and configured to prevent proximal receipt of the overtubes therein.

In an embodiment, the method further includes deploying the clip by drawing the clip proximally against the distal end of the overtubes while proximal ends of the overtubes engage distal ends of the spacers so that the clip is released from the control wires when a force exerted therein exceeds a predetermined threshold value.

In an embodiment, the clip is deployed when enlarged distal ends of the control wires received within yokes of the clip are separated from remaining lengths of the control wires.

### BRIEF DESCRIPTION

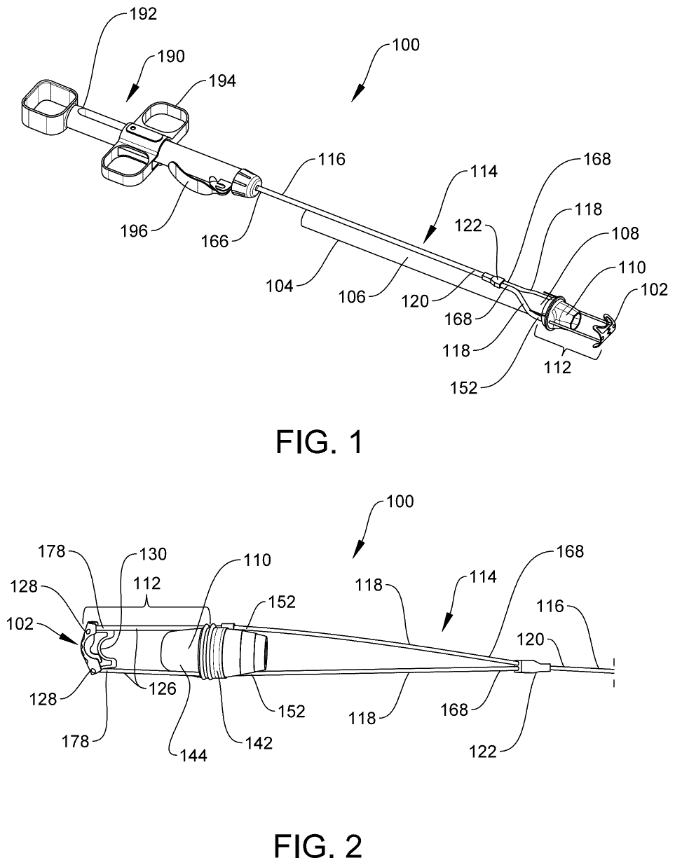

FIG. 1 shows a perspective view of an over-the-scope clipping system according to an exemplary embodiment of the present disclosure;

FIG. 2 shows a side view of a distal portion of the system according to FIG. 1;

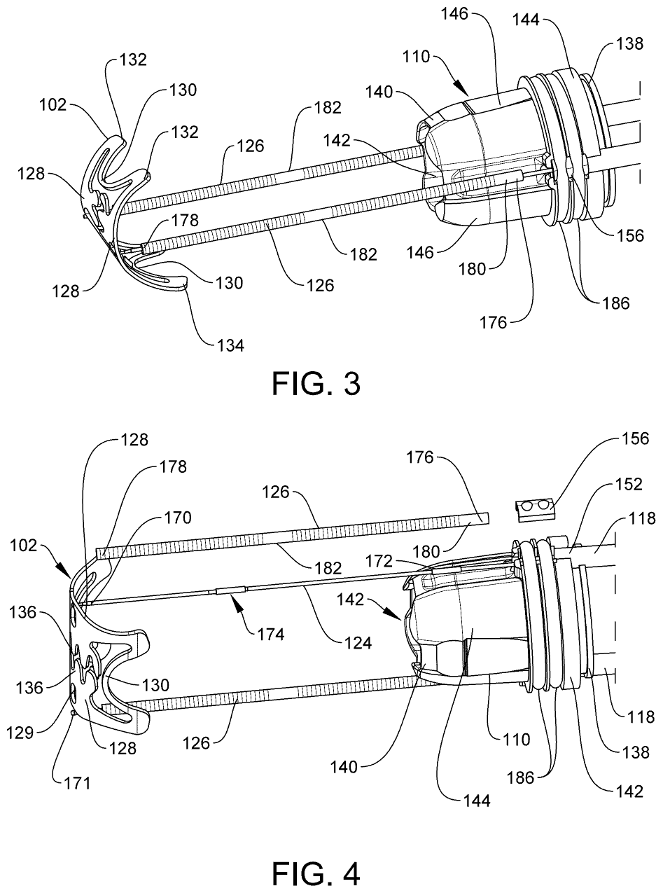

FIG. 3 shows a perspective view of the distal portion of the system according to FIG. 1;

FIG. 4 shows a partially exploded perspective view of the distal portion of the system according to FIG. 1;

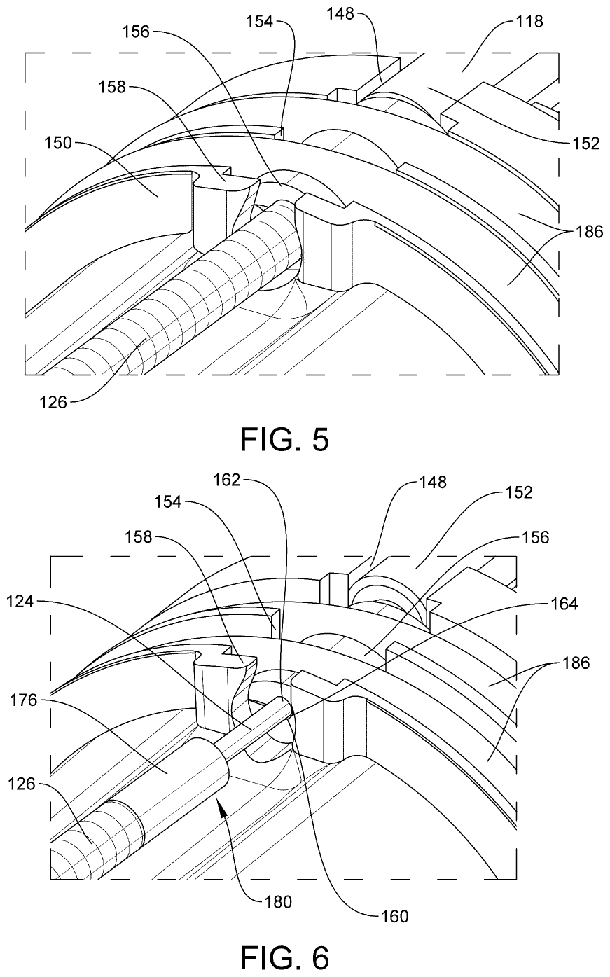

FIG. 5 shows an enlarged perspective view of a portion of an end cap and a metal spacer of the system of FIG. 1, in a review configuration;

FIG. 6 shows an enlarged perspective view of a portion of the end cap and the metal spacer of the system of FIG. 1, after a first step of a deployment process;

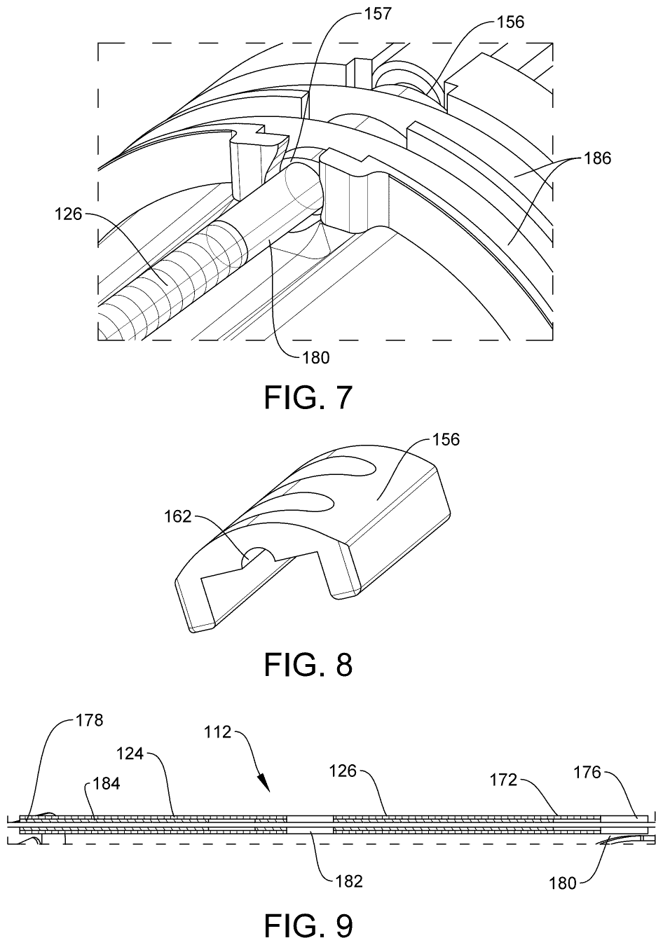

FIG. 7 shows an enlarged perspective view of a portion of the end cap and the metal spacer of the system of FIG. 1, upon initiation of a second step of the deployment process;

FIG. 8 shows a perspective view of the metal spacer according to the system of FIG. 1; and

FIG. 9 shows a longitudinal cross-sectional view of a distal portion of a control assembly according to the system of FIG. 1.

### DETAILED DESCRIPTION

The present disclosure may be further understood with reference to the following description and the appended drawings, wherein like elements are referred to with the same reference numerals. The present disclosure relates to a clipping system and, in particular, relates to an over-the-scope endoscopic clipping system, in which an initial placement of a clip may be reviewed and adjusted prior to a deployment thereof. Exemplary embodiments of the present disclosure comprise a clip mountable over a distal end of an endoscope via an end cap and movable relative to the endcap, between an insertion configuration, a closed configuration, a review configuration, and a deployed configuration via a control assembly received within a bifurcated tubing system.

The bifurcated tubing system includes a single proximal tubular structure extending along a majority of a length of the endoscope and a pair of distal tubular structures connected to a distal end of the proximal tubular structure via a bifurcation. The single proximal tubular structure reduces a footprint of the clipping system relative to clipping systems including two tubular structures extending along a full length of the endoscope, thereby improving mobility and reducing potential damage to tissue past which the endoscope is maneuvered to the target site. It will be understood by those of skill in the art that terms proximal and distal, as used herein, are intended to refer to a direction toward (proximal) and away from (distal) a user of the device.

As shown in FIGS. 1-9, a clipping system 100 for treating tissue defects and/or perforations according to an exemplary embodiment comprises a clip 102 configured to be inserted through, for example, a body lumen (accessed, e.g., via a naturally occurring bodily orifice) to a target area to clip target tissue within a living body. The clip 102 is insertable to the target area via an insertion device 104 including, for example, an endoscope 106 (e.g., a flexible endoscope). As shown in FIGS. 1-2, the clip 102 is coupled to a distal end 108 of the endoscope 106 via an end cap 110 mounted over the distal end 108 and the clip 102 is movable relative to the end cap 110 between an insertion (e.g., open) configuration, a closed configuration, a review configuration, and a deployed configuration, via a control assembly 112 to which the clip 102 is releasably coupled. The control assembly 112 is housed within a bifurcated tubing system 114 of the insertion device 104, which extends along a length of the endoscope 106 from a user interface 190 at a proximal end of the endoscope 106 to the end cap 110 at the distal end 108 of the endoscope 106.

According to an exemplary embodiment, the bifurcated tubing system 114 includes a single proximal tubular structure 116 extending form the user interface 190 along a majority of a length of the endoscope 106 and a pair of distal tubular structures 118 both of which are connected to a distal end 120 of the proximal tubular structure 116 at a bifurcation 122. The distal tubular structures 118 extend from the bifurcation 122 to opposing portions of the end cap 110 (in this illustrative embodiment, the opposing portions of the end cap 110 are diametrically opposed).

As will be described in further detail below, in an exemplary embodiment, the control assembly 112 of this embodiment includes a pair of control wires 124, each of which extends through the proximal tubular structure 116 into a corresponding one of the distal tubular structures 118 to a distal end that is releasably coupled to the clip 102. The control assembly further includes a pair of overtubes 126 received within the distal tubular structures 118 with each of the overtubes 126 extending over a corresponding one of the control wires 124 to facilitate distal movement of the clip 102 relative to the end cap 110 and a deployment of the clips 102 as will be described in more detail below.

The clip 102, as shown in FIGS. 3-4, includes a pair of jaws 128 connected to one another via hinges 130 so that the clip 102 is movable between an open configuration, in which the jaws 128 are separated from one another, and a closed configuration, in which the jaws 128 are moved toward one another to grip tissue therebetween. In an exemplary embodiment, each of the jaws 128 extends along a curve from a first end 132 to a second end 134. The curve of each of the jaws 128 in one embodiment corresponds to a curvatures of the end cap 110 over which the clip 102 is mounted in the insertion configuration. The first ends 132 of each of the jaws 128 to one another via a first one of the hinges 130, while second ends 134 of the jaws 128 are connected to one another via a second one of the hinges 130.

In an exemplary embodiment, each of the hinges 130 is a living hinge that is spring biased to bias the jaws 128 toward the closed configuration. One or more of the jaws 128 in an exemplary embodiment includes a gripping features 136 such as, for example, teeth, so that, in the closed configuration, the gripping features 136 of one of the jaws 128 contacts the gripping features 136 of the other one of the jaws 128. It will be understood by those of skill in the art, however, that the hinges 130 may have any of a variety of configurations and/or be formed of any of a variety of materials so long as the hinges 130 bias the clip 102 toward the closed configuration, as described above, and so that the bias is sufficiently strong to maintain the clip 102 in the closed configuration, gripped over a target tissue, upon deployment thereof. In an exemplary embodiment, portions of the clip 102 (e.g., hinges 130), may be formed of a shape memory alloy such as, for example, Nitinol.

It will be understood by those of skill in the art that although the clip 102 is biased toward the closed configuration, the clip 102 may be moved toward the open configuration for insertion to the target area within the body. In particular, in the insertion configuration, the clip 102 may be positioned over the end cap 110 (which will be described in further detail below) such that each of the jaws 128 extends over opposing portions of the end cap 110, an exterior surface of the end cap 110 holding the clip 102 in the open configuration. The clip 102 is configured so that, in the insertion configuration, it may be inserted through, for example, a body lumen, to the target area. When the clip 102 is positioned as desired adjacent to the target tissue, tissue is drawn into the end cap 110 between the jaws 128 of the clip 102 so that, when the clip 102 is moved distally off of the end cap 110, the clip 102 reverts toward the biased closed configuration gripping the tissue drawn into the end cap 110 between the jaws 128.

As discussed above, the clip 102 is configured (e.g., sized and shaped) to be mounted to the insertion device 104, which may include, for example, the endoscope 106. The clip 102 may be mounted to the endoscope 106 via the end cap 110, which is sized, shaped, and configured to be coupled to the distal end 108 of the endoscope 106. As will be understood by those of skill in the art, the endoscope 106 and the distal portions of the system 100 are configured to be inserted through a body lumen to a target area within the lumen and thus, must be sufficiently flexible to navigate through even tortuous paths of the body lumen.

As shown in FIGS. 3-4, the end cap 110 extends from a proximal end 138 to a distal end 140 and includes a channel 142 extending therethrough. A proximal portion 144 of the end cap 110 is configured to be mounted on or otherwise coupled to the distal end 108 of the endoscope 106 while a distal portion 146 of the end cap 110 is configured to receive the clip 102 thereover in the insertion configuration. The proximal portion 144 is coupled to the endoscope 106 so that the channel 142 of the end cap 110 of the present embodiment is substantially longitudinally aligned with a vision system and a working channel of the endoscope 106 (e.g., the distal face of the endoscope including the lens of the vision system and the distal opening of the working channel are received within the channel of the end cap 110 so that they face the open distal end of the end cap 110). Thus, tissue may be viewed through the channel 142 via an optical system of the endoscope 106.

In an exemplary embodiment, as shown in FIGS. 5-7, the proximal portion 144 of the end cap 110 of this embodiment includes a pair of holes 148 extending longitudinally (in a proximal to distal direction) through a wall 150 thereof. The holes 148 are spaced from one another around the circumference of the end cap 110 so that each of the holes 148 is positioned to receive therein a distal end 152 of a corresponding one of the distal tubular structures 118 of the bifurcated tubing system 114 adjacent to a proximal-most one of a pair of grooves 154. In an exemplary embodiment, the proximal portion 144 includes grooves 154 extending circumferentially thereabout. The holes 148 are positioned so that each of the overtubes 126 and the control wires 124 therein run distally from the corresponding tubular structure 118 across the grooves 154. Each of the grooves 154 is configured to receive a metal spacer 156 therein, between the distal end 152 of a corresponding one of the distal tubular structures 118 and a shoulder 158 at a proximal end of the wall 150 so that portions of the control assembly 112 (e.g., overtubes 126 and control wires 124) extending distally from the distal ends 152 of the distal tubular structures 118 are received between the metal spacer 156 and a portion of the grooves 154. The metal spacers 156 are held against the end cap 110, within the grooves 154 via one or more O-rings 186 which extend over the metal spacers 156, about the proximal portion 144 of the end cap 110, to press the metal spacers 156 thereagainst.

In an exemplary embodiment, each of the grooves 154 further includes a notch 160 sized and shaped to correspond to a portion of a corresponding one of the control wires 124. As shown in FIGS. 6 and 8, each of the metal spacers 156 include a corresponding notch 162 so that, when the overtubes 126 are moved distally beyond the metal spacers 156, as will be described in further detail below, the O-ring compresses the metal spacers 156 radially inward so that the notches 160, 162 together form openings 164 corresponding in size and shape to the control wires 124. While the openings 164 are configured to permit a longitudinal movement of the control wires 124 relative thereto, the openings 164 are smaller than a cross-sectional area of the overtubes 126. Thus, once the overtubes 126 have been moved distally beyond the metal spacers 156, the overtubes 126 cannot be drawn proximally back thereinto.

An outer diameter of the distal portion 146 of the end cap 110 is sized, shaped, and configured to receive the clip 102 thereover, in the insertion configuration. In an exemplary embodiment, the distal portion 146 tapers toward the distal end 140 so that the clip 102 is biased toward the closed configuration. As described above, the clip 102 is biased toward the closed configuration, but is mounted over the distal portion 146 in the insertion configuration, such that the jaws 128 extend over opposing portions thereof toward the open configuration. The clip 102 remains mounted over the end cap 110 in the open insertion configuration so long as a sufficient proximally directed tension is applied to the control wires 124. As this tension is released, the natural bias of the clip 102 draws the jaws 128 toward one another pushing the clip 102 distally over the tapered surface of the end cap 110 until the clip 102 slides distally off of the end cap 110 so that the natural bias of the clip 102 draws the clip 102 toward the closed configuration to grip tissue between the jaws 128 thereof.

As described above and as shown in FIGS. 1-2, the bifurcated tubing system 114 includes the proximal tubular structure 116 and the pair of distal tubular structures 118. The proximal tubular structure 116 extends from a proximal end 166 connected to the user interface 190 to the distal end 120. The proximal tubular structure 116 is sized, shaped, and configured to slidably receive the control wires 124 therein. In an exemplary embodiment, the proximal tubular structure 116 may be configured as a coil. It will be understood by those of skill in the art, however, that the proximal tubular structure 116 may have any of a variety of configurations so long as the proximal tubular structure 116 may be flexed along with the endoscope 106 as it being inserted through the body lumen to the target area.

Each of the distal tubular structures 118 extend from a proximal end 168 to the distal end 152. A proximal end 168 of each of the distal tubular structures 118 is connected to the distal end 120 of the proximal tubular structure 116 at the bifurcation 122 so that channels of the distal tubular structures 118 are open to and in communication with a channel of the proximal tubular structure 116. The distal ends 152 of the distal tubular structures 118 are connected to the end cap 110 via the holes 148, as described above. Each of the distal tubular structures 118 is sized and shaped to receive a corresponding one of the control wires 124 and overtubes 126 therein. It will be understood by those of skill in the art that a length of the distal tubular structures 118 corresponds in an illustrative embodiment to a desired length of the overtubes 126.

As described above, in an exemplary embodiment, the control assembly 112 includes the pair of control wires 124 and overtubes 126 extending over a portion thereof, as shown in FIG. 9. Each of the control wires 124 extends through the proximal tubular structure 116 and a corresponding one of the distal tubular structures 118 from a proximal end connected to a portion of the user interface 190 to a distal end 170 releasably coupled to the clip 102. Each of the distal ends 170 is configured to be releasably coupled to a corresponding one of the jaws 128 of the clip 102. In an exemplary embodiment, the proximal tubular structure 116 and/or the control wires 124 may include features configured to reduce the coefficient of friction therebetween. Features to reduce the coefficient of friction may include, for example, a lubricious coating or a lubricious liner extending along one of an exterior of the control wires 124 and an interior of the proximal portion 144.

In an exemplary embodiment, the control wires 124 are releasably coupled to the clip 102 via an enlarged ball 171 at the distal end 170 of each of the control wires 124, which is received within a correspondingly sized and shaped yoke 129 that is formed within or attached to a corresponding one of the jaws 128. The ball 171 may be connected to a remaining length of the control wire 124 via a joint (e.g., tapered portion, welding) configured to break, separate or otherwise release when subject to a force exceeding a predetermined threshold value. It will be understood by those of skill in the art, however, that the distal ends 170 of the control wires 124 may be releasably coupled to the clip 102 via any of a number of releasable connections so long as the clip 102 is configured to be released therefrom when the control wires 124 are subject to a force exceeding the predetermined threshold value.

Each of the control wires 124 also includes a pusher element 172 extending about a portion thereof. The pusher element 172 of this embodiment is configured as a protrusion extending radially outward from an exterior surface of the corresponding control wire 124 to interface with a portion of a corresponding one of the overtubes 126, as will be described in further detail below. The protrusion may extend about an entire periphery of the control wire 124 or, in another embodiment, extend about a portion of a periphery thereof. In one example, each of the pusher elements 172 is configured as a hypotube crimped about a corresponding one of the control wires 124. In another embodiment, each of the control wires 124 includes another protruding element 174, spaced from the pusher element 172 and positioned distally thereof to interface with a portion of a corresponding one of the overtubes 126. The protruding elements 174 are configured to interface with portions of the overtubes 126 to prevent the overtubes 126 from slipping off the control wires 124 after deployment of the clip 102. Similarly to the pusher elements 172, in one example, each of the protruding elements 174 is configured as a hypotube crimped about a portion of a corresponding one of the control wires 124.

Each of the overtubes 126 extends longitudinally from a proximal end 176 to a distal end 178 and includes a channel 184 extending therethrough. The channel 184 is sized, shaped, and configured to slidably receive a corresponding one of the control wires 124 therein. Each of the overtubes 126 is received within a corresponding one of the distal tubular structures 118 so that the overtubes 126 extend over portions of the control wires 124 received within the distal tubular structures 118. In an exemplary embodiment, each of the overtubes 126 includes a first stop 180 at the proximal end 176 thereof and a second stop 182 separated from the first stop 180 and positioned distally therefrom.

As will be described in further detail below, the first stops 180 of the overtubes 126 are configured to engage distal ends 157 of the metal spacers 156 during the deployment of the clip 102. The second stops 182 are configured to interface with the pusher elements 172 of the control wires 124 to move the clip 102 from the closed configuration, in which it is clipped over tissue, toward a review configuration in which the clip 102 is moved distally relative to the distal end 108 of the endoscope 106 by pushing the overtubes 126 distally against a surface of the clip 102. As this increases the distance between the end cap 110 and the clip 102, the field of view of the endoscope 106 is widened so that the clip 102, and the tissue gripped thereby, may be more clearly viewed via the optical/visualization system of the endoscope 106.

According to an exemplary embodiment, each of the overtubes 126 is configured as a coil, with the first and second stops 180, 182 formed via hypotube spacers, which reduce a cross-sectional area (e.g., diameter) of portions of the channels 184 of the overtubes 126 therealong. The overtubes 126 extend over the respective control wires 124 so that the pusher element 172 is positioned proximally of the second stop 182 and the protruding element 174 is positioned distally of the second stop 182. Thus, second stop 182 and the pusher element 172 are sized and shaped so that when the control wires 124 are moved distally relative to the endoscope 106, the pusher element 172 engages the second stop 182 to correspondingly move the overtubes 126 distally relative to the endoscope 106.

As described above, the distal ends 178 of the overtubes 126 engage (e.g., press distally against) a surface of the clip 102 as the overtubes 126 are moved distally relative to the endoscope 106 so that the clip 102 is correspondingly distanced from the distal end 108 of the endoscope 106. Thus, once the clip 102 has been moved from the insertion configuration toward the closed configuration, the overtubes 126 may then be moved, via the control wires 124, distally relative to the endoscope 106, to extend distally out of the distal tubular structures 118 to move the clip 102 away from the end cap 110 and the distal end of the endoscope into the review configuration.

Although the clip 102 has been described as being moved from the open insertion configuration toward the closed configuration via a release of tension along the control wires 124, it will be understood by those of skill in the art that the clip 102 may also be actively pushed distally off of the end cap 110, from the insertion configuration toward the closed configuration, by a user actuating an actuator to move the overtubes 126 distally relative to the endoscope 106. Once the clip 102 is clipped over tissue, in the closed configuration, the overtubes 126 may be moved further distally relative to the endoscope 106 to move the clip 102 from the closed configuration toward the review configuration.

It will be understood by those of skill in the art that, in the review configuration, the overtubes 126 are moved distally relative to the endoscope such that the proximal ends 176 of the overtubes 126 are not moved distally past the metal spacers 156 (see FIG. 5). Thus if, in the review configuration, a user (e.g., physician) determines that the clip 102 requires repositioning, the overtubes 126 may be drawn proximally back between the metal spacers 156 and the end cap 110 and the distal tubular structures 118 to draw the clip 102 proximally over the end cap 110 moving the clip 102 back to the insertion configuration.

In particular, to move the clip 102 from the review configuration back toward the insertion configuration to open the clip 102, the user draws the control wires 124 proximally relative to the endoscope 106. Since the control wires 124 are coupled to the clip 102, drawing the control wires 124 proximally correspondingly moves the clip 102 relative to the end cap 110 and the endoscope, and thereby the overtubes 126 which are positioned proximally thereagainst, until the jaws 128 of the clip 102 are drawn proximally against the distal portion 146 of the end cap 110. Pulling the clip 102 further proximally forces the clip 102 to open over the end cap 110 so that the clip 102 can be drawn proximally over the end cap 110 to return to the open configuration. The clip 102 may then be repositioned and once again clipped over tissue in the closed configuration and moved toward the review configuration, as described above.

Once the user has determined that the clip 102 has been clipped over target tissue as desired, the clip 102 may be moved from the review configuration toward the deployed configuration via a two-step process. In a first step, the overtubes 126 are moved further distally relative to the endoscope 106 (via the control wires 124) until proximal ends 176 of the overtubes are moved distally past the metal spacers 156 (see FIG. 6). The O-rings 186 then compress the metal spacers 156 radially inward to prevent the overtubes 126 from passing proximally between the metal spacers 156 and the end cap 110. In a second step, the control wires 124 are drawn proximally relative to the endoscope 106 so that the clip 102 is pressed proximally against the overtubes 126, causing the first stops 180 at the proximal ends 176 thereof to engage the distal ends 157 of the metal spacers 156 (see FIG. 7).

The hypotubes of the first stops 180 are configured to absorb enough compressive load as the clip 102 is drawn proximally against the distal end 178 of the overtubes 126 so that continued proximal force exerted along the control wires 124 will release the clip 102 from the control wires 124 (e.g., the balls at the distal ends 170 of the control wires 124 are released from the jaws 128) to deploy the clip 102. A position of the protruding element 174 is positioned so that, upon deployment of the clip 102, the overtubes 126 are prevented from sliding off of the control wires 124.

It will be understood by those of skill in the art that although each of the control wires 124 of the control assembly 112 is described as extending through an entire length of the bifurcating tubing system 114, from the proximal end 166 of the proximal tubular structure 116 to the distal ends 152 of the distal tubular structures 118, in an alternate embodiment, each of the control wires 124 extends through a distal portion of the proximal tubular structure 116 and a corresponding one of the distal tubular structures 118 to connect to the clip 102. In this embodiment, proximal ends of the control wires 124 are connected to a single proximal control member slidably received within the proximal tubular structure 116 so that moving the single control member longitudinally relative to the endoscope 106 correspondingly moves the control wires 124, as desired.

According to an exemplary embodiment, as shown in FIG. 1, the user interface 190 includes a handle shaft 192 connected to the proximal end of the endoscope 106 and a slider 194 slidable longitudinally thereover. Proximal ends of the control wires 124 are connected to the slider 194 so that moving the control wires 124 proximally and distally relative to the handle shaft 192 correspondingly moves the control wires 124 proximally and distally, respectively, relative to the endoscope 106.

As described above, the control wires 124 may be moved distally relative to the endoscope 106 to correspondingly move the overtubes 126 and the clip 102 distally relative to the endoscope 106 from the open insertion configuration to the closed configuration, from the closed configuration to the review configuration, and from the review configuration to the first step of the deployed configuration. The slider 194 is moved proximally relative to the handle shaft 192 to correspondingly move the control wires 124 from the review configuration to the insertion configuration, or from the first step of the deployed configuration to the second step of the deployed configuration.

In an exemplary embodiment, the user interface 190 includes a lock 196 movable between a locked configuration, in which the slider 194 is prevented from being moved further distally relative to the handle shaft 192 (i.e., from the review configuration toward the first step of deployment), and an unlocked configuration, in which the slider 194 is permitted to be moved distally relative to the shaft 192 so that the overtubes 126 may be moved distally past the metal spacers 156 toward the first step of the deployed configuration.

According to an exemplary method for tissue closure utilizing the clipping system 100, the clip 102 is inserted through a body lumen such as, for example, the GI tract, to a target area within the body lumen via the insertion device which, in an exemplary embodiment, may include the endoscope 106. As described above, in the insertion configuration, the clip 102 is mounted to the distal end 108 of the endoscope 106 via the end cap 110 so that jaws 128 of the clip 102 are separated from one another in the open configuration. The clip 102 is guided to the target area via the visualization system of the endoscope and positioned over a target tissue.

A suction force and/or tissue graspers may be applied through a working channel of the endoscope 106 so that the target tissue may be drawn into the channel 142 of the end cap 110 and between the jaws 128. Thus, when the clip 102 is moved distally off of the end cap 110 by releasing tension along the control wires 124 (or by moving the overtubes 126 distally relative to the endoscope 106 via a distal movement of the control wires 124), the clip 102 is permitted to revert toward the biased closed configuration to grip tissue between the jaws 128.

It will be understood by those of skill in the art that suctioning and/or gripping of the tissue in the closed configuration may obstruct an imaging/optical lens of the endoscope 106 so that the user is unable to visualize and/or confirm whether a desired target tissue has been properly clipped. Thus, upon an initial clipping of the tissue, the clip 102 may be moved toward the review configuration by moving the control wires 124 distally relative to the endoscope 106 (e.g., by moving the slider 194 distally relative to the handle shaft 192) such that the pusher element 172 thereof engages the second stop 182 to move the overtubes 126 distally relative to the endoscope 106, thereby moving the clip 102 away from the end cap 110.

It will be understood by those of skill in the art that as the overtubes 126 are pushed distally against the clip 102, the clip 102 may be moved distally away from the endoscope 106 to distance the clip 102 from the end cap 110 in the review configuration. The increased distance between the end cap 110 and the clip 102 widens a field of view of the endoscope 106 so that the clip 102, and the tissue gripped thereby, may be viewed via the optical/visualization system of the endoscope 106. In this review configuration, the user may determine whether the clip 102 needs to be repositioned or whether the clip 102 is clipped over the target tissue as desired, and thus ready to be deployed.

If the user determined that the clip 102 should be repositioned, the control wires 124 are drawn proximally relative to the endoscope 106 so that the clip 102 which is coupled to the distal end 170 of the control wires 124, is drawn toward the endoscope 106 (or rather, the endoscope 106 is advanced distally toward the clip 102 as the clip 102 remains coupled to the tissue) by, for example, drawing the slider proximally relative to the handle shaft 192. The clip 102 is drawn proximally so that the jaws 128 are moved over the distal portion of the end cap 110 toward the open configuration, releasing the tissue that had been gripped therebetween. The distal end 108 of the endoscope 106 may then be repositioned and different tissue may be suctioned and/or received within the channel 142 of the end cap 110. The clip 102 may then be once again moved from the open configuration (e.g., the insertion configuration) to the closed configuration to grip the tissue between the jaws 128. The clip 102 is then again moved to the review configuration so that the user may determine whether a desired placement of the clip 102 has been achieved.

This process is repeated until it is determined that the clip 102 is clipped over the target tissue in a desired configuration. The user may then initiate the first step of the deployment process by moving the overtubes 126 further distally relative to the endoscope 106 until the proximal ends 176 are moved distally past the distal end of the metal spacers 156. This permits the metal spacers 156 to compress radially inward over the control wires 124 proximally of the overtubes 126 preventing the overtubes 126 from being drawn proximally back between the metal spacers 156 and the end cap 110.

In the second step of the deployment process, the control wires 124 are drawn proximally relative to the endoscope 106 so the clip 102 is drawn proximally against the distal ends 178 of the overtubes 126, as the first stops 180 at the proximal ends 176 of the overtubes 126 abut against and engage the distal ends 157 of the metal spacers 156. Continued proximal force applied to the control wires 124 at this point increases the tension on the control wires 124 until the force exerted thereon exceeds a predetermined threshold value and the clip 102 is released from the distal ends 170 of the control wires 124—e.g., the ball 171 at each of the distal end 170 of each of the control wires 124 is separated from a remaining length thereof. Thus, the insertion device 104, including the control wires 124 and the overtubes 126, may be withdrawn proximally away from the clip 102 and out of the body, leaving the clip 102 clipped over the target tissue in the deployed configuration. As described above, the protruding elements 174 of the control wires 124 are drawn proximally against the second stops 182 of the overtubes 126 so that the overtubes 126 are prevented from sliding off of the control wires 124 as the insertion device 104 is being removed from the body.

It will be apparent to those skilled in the art that various modifications may be made in the present disclosure, without departing from the scope of the disclosure. Furthermore, those skilled in the art will understand that the features of any of the various embodiments may be combined in any manner that is not inconsistent with the description and/or the functionality of the embodiments.

1-15. (canceled)

16. A clipping system for treating tissue, comprising:
an end cap including a proximal portion configured to be coupled to a distal end of an insertion device and a distal portion extending distally from the proximal portion;
a clip including first and second jaws movable relative to one another between an open configuration, in which the first and second jaws are separated from one another, and a biased closed configuration, in which the first and second jaws are moved toward one another, the clip configured to be mounted over the distal portion of the end cap in the open configuration and;
a bifurcated tubing system including a single proximal tubular structure extending from a proximal end to a distal end and a pair of distal tubular structures, each of which extends from a proximal end to a distal end, the proximal end of each of the distal tubular structures connected to the distal end of the single proximal tubular structure via a bifurcation such that the pair of distal tubular structures are in communication with the single proximal tubular structure, the distal end of the each of the distal tubular structures connected to the end cap; and
a control assembly including a pair of control wires, each of which extends through the single proximal tubular structure and a corresponding one of the distal tubular structures to be releasably coupled to the clip, and a pair of overtubes received within the distal tubular structures to extend over a portion of the control wires received therein, the control wires and the overtubes configured to interface with one another and be longitudinally movable relative to the end cap to move the clip between an insertion configuration, in which the clip is mounted over the end cap in the open configuration, the closed configuration, in which the clip is moved distally off of the end cap to grip tissue therebetween, and a review configuration, in which the clip is physically separated from the end cap to enhance visual observation of the clip.

17. The system of claim 16, wherein the proximal portion of the end cap includes a pair of longitudinally extending grooves aligned with the distal tubular structures, a spacer received within each of the grooves and held thereagainst via an O-ring.

18. The system of claim 17, wherein the spacers are movable relative to the end cap from a first position, in which the overtubes are slidably receivable between the grooves and the spacers, and a second position, in which the O-ring compresses the spacer radially inward upon movement of the overtubes distally therepast so that openings formed between the spacers and the grooves are sized, shaped and configured to prevent proximal receipt of the overtubes therein.

19. The system of claim 18, wherein each of the overtubes includes a first stop configured to engage a distal end of a corresponding one of the spacers when the corresponding one of the spacers is in the second position and a second stop configured to interface with a portion of the corresponding one of the control wires.

20. The system of claim 19, wherein each of the control wires includes a pusher element protruding from the control wire, the control wire received within the corresponding overtube so that the pusher element is positioned proximally of the second stop so that the pusher element engages the stop as the control wire is moved distally relative to the end cap to correspondingly move the overtube distally relative to the end cap.

21. The system of claim 19, wherein each of the control wires includes a protruding element along a portion of the control wire, and the control wire is received within the corresponding overtube so that the protruding element is positioned distally of the second stop to prevent the corresponding overtube from slipping thereoff of subsequent to a deployment of the clip.

22. The system of claim 20, wherein one of the pusher element and the protruding element is configured as a hypotube crimped over a portion of the control wire.

23. The system of claim 16, wherein each of the control wires is releasably coupled to a corresponding one of the first and second jaws of the clip via an enlarged distal end received within a yoke of the corresponding one of the first and second jaws, the enlarged distal end connected to a remaining length thereof via a joint configured to separate the enlarged distal end from the remaining length when subject to a force exceeding a predetermined threshold value.

24. The system of claim 23, wherein each of the enlarged distal ends has a cross-sectional area that is larger than a cross-sectional area of an opening through which it enters the yoke of the corresponding jaw.

25. A clipping system for treating tissue, comprising:
an end cap including a proximal portion couped to a distal end of an endoscope and a distal portion extending distally from the proximal portion;
a clip mounted over the distal portion of the end cap so that first and second jaws of the clip are separated from one another toward an open configuration, the clip biased toward a closed configuration, in which the first and second jaws are moved toward one another;
a bifurcated tubing system including a single proximal tubular structure and a pair of distal tubular structures, the single proximal tubular structure extends along a majority of a length of the endoscope from a proximal end connected to a user interface at a proximal end of an insertion device to a distal end, proximal ends of the distal tubular structures connected to the distal end of the single proximal tubular structure via a bifurcation such that the distal tubular structures are in communication with the single proximal tubular structure and distal ends of the distal tubular structures connected to the end cap; and
a control assembly including a pair of control wires and a pair of overtubes, the control wires extending through the single proximal tubular structure and the distal tubular structures so that distal ends of the control wires extend distally past the distal ends of the distal tubular structures to be releasably coupled to the clip, the overtubes received within the distal tubular structures to extend over portions of the control wires received therein, the control wires and the overtubes interfacing with one another and movable relative to the insertion device to move the clip relative to the end cap between an insertion configuration, in which the clip is mounted over the end cap, the closed configuration, in which the clip is slid off of the end cap to grip tissue between the first and second jaws thereof, and a review configuration, in which the clip is physically separated from the end cap to enhance visual observation of the clip.

26. The system of claim 25, wherein the proximal portion of the end cap includes a pair of longitudinally extending grooves aligned with the distal tubular structures, spacers received within the grooves and held thereagainst via O-rings which moves the spacers from a first position, in which the overtubes is slidably receivable between the grooves and the spacers, and a second position, in which the O-rings compress the spacers radially inward upon movement of the overtubes distally therepast so that openings formed between the spacers and the grooves are sized, shaped and configured to prevent proximal receipt of the overtubes therein.

27. The system of claim 26, wherein each of the overtubes include a first stop configured to engage a distal end of a corresponding one of the spacers when the corresponding one of the spacers is in the second position and a second stop configured to interface with a portion of the corresponding one of the control wires.

28. The system of claim 27, wherein each of the control wires includes a pusher element protruding from the control wire, the control wire received within the corresponding overtube so that the pusher element is positioned proximally of the second stop so that the pusher element engages the stop as the control wire is moved distally relative to the end cap to correspondingly move the overtube distally relative to the end cap.

29. The system of claim 28, further comprising:
a user interface including a handle shaft connected to a proximal end of the insertion device and a slider slidably mounted over the handle shaft and connected to proximal end of the control wires so that a longitudinal movement of the slider relative to the handle shaft correspondingly moves the control wires relative to the endoscope.

30. The system of claim 29, wherein the user interface further includes a lock movable between the first position, in which the slider is prevented from being moved further distally relative to the handle shaft to prevent movement of the clip from the review configuration toward a deployed configuration, and the second position, in which the slider is distally movable relative to the handle shaft to initiate the deployed configuration in which the overtube is moved distally past the spacer.

31. A method for treating tissue, comprising:
inserting a clip to a target area in a body lumen via an endoscope, the clip mounted over a distal end of the endoscope, via an adapter, in an open insertion configuration in which jaws of the clip are separated from one another;
drawing tissue into a channel of the adapter and between the jaws of the clip; moving the clip from the open insertion configuration toward a biased closed configuration by releasing a tension along a pair of control wires, distal ends of which are releasably coupled to the jaws of the clip, the control wires extending through a bifurcated tubing assembly including a single proximal tubular structure extending along a majority of a length of the endoscope and a pair of distal tubular structures connected to the single proximal tubular structure via a bifurcation;
moving the clip from the closed configuration toward a review configuration by moving the clip distally relative to the adapter so that the clip is spaced therefrom and visible via a viewing system of the endoscope, the clip moved toward the review configuration via overtubes moved distally relative to the endoscope to be pressed distally against a surface of the clips, the overtubes received within the distal tubular structures and extending over portions of the control wires received therein, interfacing therewith such that a distal motion of the control wires relative to the endoscope correspondingly moves the overtubes distally relative to the endoscope; and
determining whether the clip is clipped over a target tissue, as desired.

32. The method of claim 31, wherein, when a user determines that the clip requires repositioning, drawing the control wires proximally relative to the endoscope to draw the clip toward an end cap and over a distal portion thereof to the open insertion configuration.

33. The method of claim 31, wherein, when a user determines that the clip is clipped over the target tissue, initiating a deployment of the clip by moving the overtubes further distally relative to the endoscope so that the overtubes are moved distally past spacers housed against portions of an end cap via O-rings, the O-rings compressing the spacers radially so that openings formed between the spacers and are sized, shaped and configured to prevent proximal receipt of the overtubes therein.

34. The method of claim 33, further comprising deploying the clip by drawing the clip proximally against the distal end of the overtubes while proximal ends of the overtubes engage distal ends of the spacers so that the clip is released from the control wires when a force exerted therein exceeds a predetermined threshold value.

35. The method of claim 34, wherein the clip is deployed when enlarged distal ends of the control wires received within yokes of the clip are separated from remaining lengths of the control wires.
